(19) United States
(12) Patent Application Publication
(10) Pub. No.: US 2021/0323949 A1
(43) Pub. Date: Oct. 21, 2021

(54) COMPOUNDS AND METHODS FOR INHIBITION OF MULTIPLE MYELOMA

(71) Applicants: FLORIDA ATLANTIC UNIVERSITY BOARD OF TRUSTEES, BOCA RATON, FL (US); H. Lee Moffitt Cancer Center and Research Institute, Inc., Tampa, FL (US)

(72) Inventors: Gregg B. Fields, Palm Beach Gardens, FL (US); Conor Lynch, Tampa, FL (US)

(73) Assignees: FLORIDA ATLANTIC UNIVERSITY BOARD OF TRUSTEES, Boca Raton, FL (US); H. Lee Moffitt Cancer Center and Research Institute, Inc., Tampa, FL (US)

(21) Appl. No.: 17/272,135

(22) PCT Filed: Jun. 28, 2019
(86) PCT No.: PCT/US2019/039797

§ 371 Date: Feb. 26, 2021

Related U.S. Application Data
(60) Provisional application No. 62/724,828, filed on Aug. 30, 2018.

(51) Int. Cl. C07D 405/14; A61P 35/00; C07D 405/12

(52) U.S. Cl. CPC C07D 405/14; C07D 405/12; A61P 35/00

(57) ABSTRACT

Novel compounds (small molecules) that potently and selectively inhibit MMP-13 (i.e., MMP-13 inhibitors) are used for treatment of multiple myeloma. The MMP-13 inhibitors described herein are highly selective for MMP-13 and when administered to an individual in need thereof, the compounds selectively kill multiple myeloma cells, reduce growth of multiple myeloma cells, inhibit multiple myeloma-induced osteoclastogenesis, and increase survival time in the individual.

### CROSS-REFERENCE TO RELATED APPLICATION

This application claims priority to U.S. Provisional Application No. 62/724,828 filed Aug. 30, 2018, which is herein incorporated by reference in its entirety.

### STATEMENT REGARDING FEDERALLY SPONSORED RESEARCH

This invention was made with government support under grant number AR063795 awarded by the National Institutes of Health. The government has certain rights in the invention.

### FIELD OF THE INVENTION

The invention relates generally to the fields of pharmacology, medicine, and oncology. In particular, the invention relates to novel compounds for treating multiple myeloma.

### BACKGROUND

Multiple myeloma will prove to be fatal for over 12,500 American men and women in 2018. During disease progression, myeloma colonizes the skeleton and causes extensive bone destruction leading to intense pain and pathologic fracture that greatly contribute to patient morbidity (Rage N and Roodman G D, Clin Cancer Res (2011) 17: 1278-1286). While the advent of therapies such as proteasome inhibitors (bortezomib/carfilzomib), chemotherapies (melphalan) and immunomodulators (thalidomide) have improved outcomes for multiple myeloma patients, the average survival time is 5-6 years following diagnosis of active disease (Laubach et al., Seminars in Oncology (2013) 40: 549-553; Shay et al., J Mol Med (2016) 94: 21-35). Therefore, diagnostics and therapies that can identify patients at high-risk for progression or significantly impact myeloma growth are an urgent and unmet clinical need for this currently incurable disease.

Matrix metalloproteinase 13 (MMP-13) is an interstitial collagenase widely expressed in the skeleton where it has noted roles in endochondral ossification. In the context of multiple myeloma, MMP-13 expression by myeloma cells has been demonstrated with serum levels of MMP-13 increased in patients with bone disease (Fu et al., J Clin Invest (2016) 126: 1759-1772). Abundant MMP expression is found at the cancer-bone interface where MMPs play roles in extracellular matrix (ECM) remodeling and the bioactivity/availability of factors such as TGFβ. Of the MMPs identified, MMP-13 was the most upregulated, and it is mainly expressed by bone building mesenchymal stromal cells (MSCs) and osteoblasts but not by bone resorbing osteoclasts.

Recent studies have produced a variety of selective MMP-13 inhibitors (Xi et al., Chem Med Chem (2017)12:1157-1168). However, distinct drawbacks to these inhibitors have been reported. For inhibitors presented as organic anions, binding to human organic anion transporter 3 resulted in nephrotoxicity (Ruminski et al., J Med Chem (2016)59:313-327). Inhibitors possessing carboxylic acids may generate reactive metabolites through protein conjugation of the resulting acyl glucuronide (Ruminski et al., J Med Chem (2016)59:313-327; Sallusti et al., Curr Drug Metab (2000)1:163-180). Pyrimidine-2-carboxamide-4-one-based inhibitors have exhibited poor bioavailability, low volume of distribution, poor metabolic stability, and/or P450 3A4 inhibition (Nara et al., Bioorg Med Chem (2016)24:6149-6165). Obtaining appropriate kinetic solubilities for MMP-13 inhibitors has proved challenging (Nara et al., Bioorg Med Chem (2014)22:5487-5505; Spicer et al., J Med Chem (2014)57:9598-9611). Some of the most promising recent selective MMP-13 inhibitors displayed poor solubility, permeability, biodistribution, metabolic stability, and/or bioavailability. There is thus a need for new and efficacious MMP-13 inhibitors.

### SUMMARY

Described herein are novel, potent, and selective MMP-13 inhibitor compounds that avoid the drawbacks of the prior art inhibitors, particularly poor solubility and metabolic stability as well as the potential for nephrotoxicity and generation of reactive metabolites. In the experiments described below, the potent and selective MMP-13 inhibitors indicate a role for MMP-13 proteolytic activity in the progression of multiple myeloma. Because MMP-13 is critical for multiple myeloma progression, the selective MMP-13 inhibitors described herein are useful for treatment of multiple myeloma.

Accordingly, described herein is a compound of Formula A:

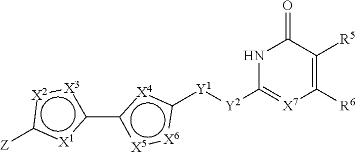

wherein
group Z is of formula C(═O)NHCH(R^{2A})C(═O)NHR^{2B};
R^{2A} is (C₁-C₄)alkyl or (C₃)cycloalkyl, and R^{2B} is 4-membered heterocyclyl or CH₃;

### X¹ is O;

X² and X³ are each independently CR³;
such that the ring comprising X¹, X², and X³ is heteroaryl;
R³ is independently at each occurrence H;
X⁴ is C(R⁴)═C(R⁴);
X⁵ and X⁶ are each independently CR⁴;
such that the ring comprising X⁴, X⁵, and X⁶ is aryl;
R⁴ is independently at each occurrence H or F;

### Y¹ is CHR;

### Y² is S, CHR, or NR;

### X⁷ is N;

### R is H;

R⁵ and R⁶ together with the ring carbon atoms to which they are bonded together form a 5-membered cycloalkyl ring;
or a pharmaceutically acceptable salt thereof. The compound inhibits multiple myeloma cell growth. In one embodiment of the compound, X⁵ and X⁶ are both CR⁴. In another embodiment of the compound, X¹ is O and X⁴ is CH═CH. In another embodiment of the compound, X¹ is O, X² and X³ are both CH, and R⁴ is H or F. In another embodiment of the compound, the compound has the formula:

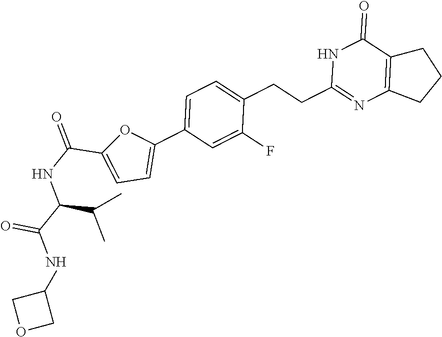 [SMILES: CC(C)[C@H](NC(=O)c1ccc(-c2ccc(CCc3nc4c(c(=O)[nH]3)CCC4)c(F)c2)o1)C(=O)NC1COC1]

In another embodiment of the compound, the compound has the formula:

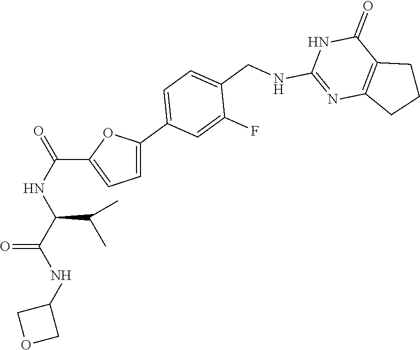 [SMILES: CC(C)[C@H](NC(=O)c1ccc(-c2ccc(CNc3nc4c(c(=O)[nH]3)CCC4)c(F)c2)o1)C(=O)NC1COC1]

In another embodiment of the compound, the compound has the formula:

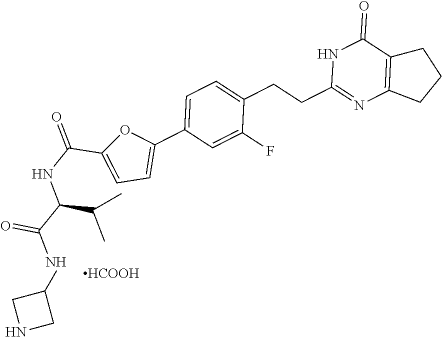 [SMILES: CC(C)[C@H](NC(=O)c1ccc(-c2ccc(CCc3nc4c(c(=O)[nH]3)CCC4)c(F)c2)o1)C(=O)NC1CNC1 ; "·HCOOH"]

In another embodiment of the compound, the compound has the formula:

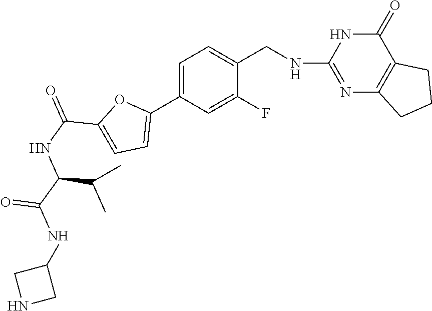 [SMILES: CC(C)[C@H](NC(=O)c1ccc(-c2ccc(CNc3nc4c(c(=O)[nH]3)CCC4)c(F)c2)o1)C(=O)NC1CNC1]

Also described herein is a composition including any of the above compounds and a pharmaceutically acceptable carrier.

Further described herein is a method of treating multiple myeloma in an individual (e.g., a human). The method includes administering to the individual any of the above compounds or a composition including any of the above compounds in a therapeutically effective amount to reduce at least one of: MMP-13 concentration and MMP-13 proteolytic activity in the individual. In one embodiment of the method, the compound has the formula:

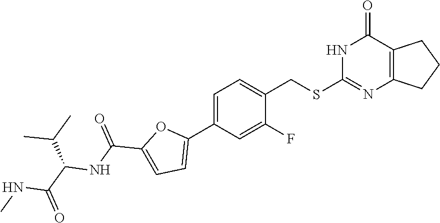 [SMILES: CNC(=O)[C@@H](NC(=O)c1ccc(-c2ccc(CSc3nc4c(c(=O)[nH]3)CCC4)c(F)c2)o1)C(C)C]

In the method, administering the compound or composition selectively kills multiple myeloma cells in the individual. In the method, administering the compound or composition reduces growth of multiple myeloma cells in the individual. In the method, administering the compound or composition inhibits multiple myeloma-induced osteoclastogenesis in the individual. In the method, administering the compound or composition increases survival time in the individual. In some embodiments of the method, the individual is considered high-risk for progression of the multiple myeloma. The method can further include detecting a state or condition of multiple myeloma in the individual prior to administering the compound or the composition to the individual.

The terms “group,” “functional group,” “moiety,” “molecular moiety,” or the like are somewhat synonymous in the chemical arts and are used to refer to distinct, definable portions or units of a molecule, and to units that perform some function. Examples of functional groups that are suitable for the compounds described herein include, but are not limited to, aryl or heteroaryl group, alkyl, cycloalkyl or the like.

As used herein, the term “alkyl” refers to a saturated hydrocarbon fragment. For example, in one embodiment, an alkyl can be a saturated hydrocarbon moiety containing up to six carbons (e.g., methyl, ethyl).

As used herein, the term “cycloalkyl groups” are groups containing one or more carbocyclic rings including, but not limited to, cyclopropyl, cyclobutyl, cyclopentyl, cyclohexyl, cycloheptyl, and cyclooctyl groups.

As used herein, the term “aryl groups” refers to cyclic aromatic hydrocarbons that do not contain heteroatoms in the ring. An aromatic compound, as is well-known in the art, is a multiply-unsaturated cyclic system that contains 4n+2π electrons where n is an integer.

As used herein, the term “heteroaryl” refers to aromatic cycles where one or more heteroatoms form part of the ring. The heteroaryl ring may also be substituted with a variety of functional and/or alkyl groups (e.g., C₁-C₆ alkyl).

By the term “osteoclastogenesis” is meant the development of osteoclasts, which are cells that break down bone. By “multiple myeloma-induced osteoclastogenesis” is meant the development of osteoclasts as influenced by multiple myeloma cells.

The term “purified” means separated from many other entities (small molecules, compounds, proteins, nucleic acids), and does not require the material to be present in a form exhibiting absolute purity, exclusive of the presence of other entities. In some embodiments, a small molecule, compound, protein, nucleic acid or other entity is considered pure (purified) when it is removed from substantially all other entities.

By the terms “to modulate” and “modulates” is meant to increase or decrease. These terms can refer to increasing or decreasing an activity, level or function of a molecule (e.g., protein, peptide, nucleic acid, small molecule, metabolite), or effecting a change with respect to one or more biological or physiological mechanisms, effects, responses, functions, pathways or activities in which, for example, osteoclastogenesis is involved.

The terms “agent” and “therapeutic agent” as used herein refer to a chemical entity or biological product, or combination of chemical entities or biological products, administered to a subject (a mammal such as a human) to treat a disease or condition (e.g., multiple myeloma). Examples of therapeutic agents include small molecules (compounds) and biologics, which may be referred to herein as a “drug” or “therapeutic drug”.

The terms “patient,” “subject” and “individual” are used interchangeably herein, and mean a subject, typically a mammal, to be treated, diagnosed, and/or to obtain a biological sample from. Subjects include, but are not limited to, humans, non-human primates, horses, cows, sheep, pigs, rats, mice, insects, dogs, and cats. A human in need of multiple myeloma treatment is an example of a subject.

The terms “sample,” “patient sample,” “biological sample,” and the like, encompass a variety of sample types obtained from a patient, individual, or subject and can be used in a therapeutic drug screening, diagnostic or monitoring assay. The patient sample may be obtained from a healthy subject, a diseased patient or a patient having associated symptoms of a particular disease or disorder (e.g., multiple myeloma). Moreover, a sample obtained from a patient can be divided and only a portion may be used for therapeutic drug screening. Further, the sample, or a portion thereof, can be stored under conditions to maintain sample for later analysis. The definition encompasses blood and other liquid samples of biological origin (including, e.g., urine, plasma, serum, peripheral blood), bone marrow, biopsy specimens or tissue cultures or cells derived therefrom and the progeny thereof. In a specific embodiment, a sample includes a plasma sample. In another embodiment, a urine sample is used.

As used herein, the terms “therapeutic treatment” and “therapy” are defined as the application or administration of a therapeutic agent (e.g., an MMP-13 inhibitor as described herein) or therapeutic agents to a patient who has a disease, a symptom of disease or a predisposition toward a disease, with the purpose to cure, heal, alleviate, relieve, alter, remedy, ameliorate, improve or affect the disease, the symptoms of disease, or the predisposition toward disease.

Although compounds, compositions, methods and kits similar or equivalent to those described herein can be used in the practice or testing of the present invention, suitable compounds, compositions, methods and kits are described below. All publications, patent applications, and patents mentioned herein are incorporated by reference in their entirety. In the case of conflict, the present specification, including definitions, will control. The particular embodiments discussed below are illustrative only and not intended to be limiting.

### BRIEF DESCRIPTION OF THE DRAWINGS

FIGS. 1A, 1B and 1C: FIG. 1A shows MMP-13 specific inhibitor RF-036 (coded as SR-18465, compound (S)-17b in Choi et al., J Med Chem (2017)60:3814-3827). The IC₅₀ value was calculated using a fluorogenic triple-helical substrate. The structure for RF-036 is also illustrated in FIG. 1A. FIGS. 1B and 1C show results from an experiment in which bone marrow-derived macrophages (BMMs) were treated with varying concentrations of the MMP-13 inhibitor RF-036, M-CSF, and RANKL (receptor activator of nuclear kappa-β ligand) at day 0 and the number of tartrate-resistant acidic phosphatase (TRAP)-positive osteoclasts (FIG. 1B; arrows) per well counted (FIG. 1C) after 5 days. Asterisks denote statistical significance (p<0.001).

FIG. 2 is a graph showing results from an experiment in which MMP-13 specific inhibitor RF-036 was added to mature osteoclasts and the number of TRAP positive osteoclasts per well counted after 5 days.

FIG. 3 is a pair of graphs showing results from an experiment in which BMMs were treated with varying concentrations of the MMP-13 inhibitors RF-040 (compound (S)-17c in Choi et al., J Med Chem (2017)60:3814-3827) or RF-334 (compound 52 in Fuerst et al., Bioorg Med Chem (2018)26:4984-4995), M-CSF, and RANKL at day 0 and the number of TRAP-positive osteoclasts per well counted after 5 days; below the graphs are chemical formulas for RF-040 and RF-334.

FIG. 4A and FIG. 4B are graphs showing experimental results of RF-036 impact on the proliferation of (FIG. 4A) multiple myeloma cell lines, (FIG. 4B) MSCs, and (FIG. 4B) monocytic cells (RAW 267 commonly used as osteoclast precursor). Asterisks denote statistical significance.

FIG. 5A is a set of images and FIG. 5B and FIG. 5C are graphs showing the efficacy of RF-036 (MMP-13i) for the treatment of multiple myeloma. FIG. 5A, 5B: Mice (C57BL/6 KalwRij) were inoculated with 5TGM1 luciferase expressing multiple myeloma cells (1×10⁶) and after 10 days randomized into vehicle (n=7) or MMP-13i (n=6) groups. Mice were treated daily with RF-036 at 2 mg/kg via intraperitoneal injection. Bioluminescence (FIG. 5A) was quantitated every 3-4 days (FIG. 5B). Figure C: IgG2b measurements in serum (as a readout for tumor burden) also showed significant differences between the vehicle and RF-036 groups at day 28. Asterisks denote statistical significance.

FIG. 6 is a Kaplan-Meier curve of overall survival in wild type and MMP-13 null animals. Wild type or MMP-13 null animals (n=10/group) were inoculated with luciferase expressing 5TGM1 cells. Overall survival in the MMP-13 null multiple myeloma bearing mice was significantly higher than controls with median survival times of 43 and 39 days, respectively (p=0.0011). While on the surface, this does not appear to be a large difference it is impressive given the rapidity and aggressiveness of the 5TGM1 model. Furthermore, the observed difference in overall survival time is in keeping with standard of care therapies tested in the 5TGM1 model and the related 5T2MM model such as bortezomib, bisphosphonates, and melphalan.

FIG. 7 is a Kaplan-Meier curve of overall survival time of 5TGM1 multiple myeloma bearing mice treated with RF-036 (MMP-13i) (n=6; 2 mg/kg daily) or vehicle control (n=7). The median survival time for the RF-036 group was 46 days compared to 41 for the vehicle control group.

FIG. 8 is Scheme 1 illustrating assembly of the key intermediate for the eventual synthesis of GF-01 and GF-03.

FIG. 9 is Scheme 2 in which assembly of GF-01 (Target-1) and GF-03 (Target-2) is illustrated.

FIG. 10 is Scheme 3 in which assembly of GF-02 (Target-3) and GF-04 (Target-4) is illustrated.

### DETAILED DESCRIPTION

Described herein are novel compounds (small molecules) that potently and selectively inhibit MMP-13 (i.e., MMP-13 inhibitors) for use in treatment of multiple myeloma. A role for MMP-13 catalytic activity in multiple myeloma was discovered. The MMP-13 inhibitors described herein are highly selective for MMP-13 with IC₅₀ values <100 nM.
MMP-13 Inhibitor Compounds and Compositions Thereof

An MMP-13 inhibitor as described herein is any compound of formula A:

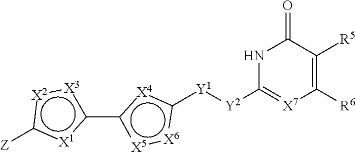

wherein

group Z is of formula C(═O)NHCH(R^{2A})C(═O)NHR^{2B};

R^{2A} is (C₁-C₄)alkyl or (C₃)cycloalkyl, and R^{2B} is 4-membered heterocyclyl or CH₃;

X¹ is O;

X² and X³ are each independently CR³;

such that the ring comprising X¹, X², and X³ is heteroaryl;

R³ is independently at each occurrence H;

X⁴ is C(R⁴)═C(R⁴);

X⁵ and X⁶ are each independently CR⁴;

such that the ring comprising X⁴, X⁵, and X⁶ is aryl;

R⁴ is independently at each occurrence H or F;

Y¹ is CHR;

Y² is S, CHR, or NR;

X⁷ is N;

R is H;

R⁵ and R⁶ together with the ring carbon atoms to which they are bonded together form a 5-membered cycloalkyl ring;

or a pharmaceutically acceptable salt thereof.

In one embodiment of a compound of Formula A, X⁵ and X⁶ are both CR⁴.

In one embodiment of a compound of Formula A, X¹ is O and X⁴ is CH═CH.

In one embodiment of a compound of Formula A, X¹ is O, X² and X³ are both CH, and R⁴ is H or F.

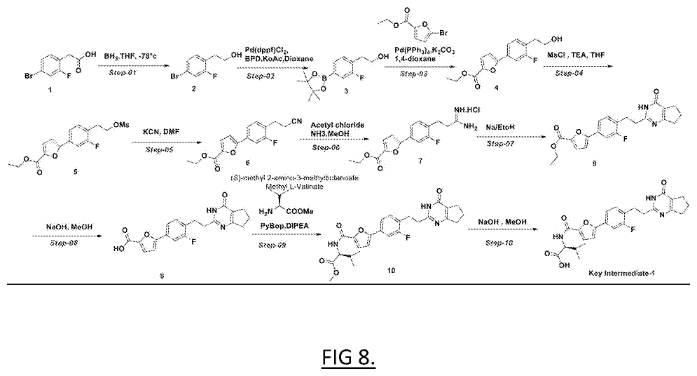

The MMP-13 inhibitor compounds described herein were synthesized by using the synthetic route described (compound (S)-17b) in Choi et al., J Med Chem (2017)60:3814-3827) for RF-036 or outlined in Schemes 1-3 for compounds GF-01, GF-02, GF-03, and GF-04. In Scheme 1 (FIG. 8), assembly of the key intermediate for the eventual synthesis of GF-01 and GF-03 is described.

GF-01 inhibits MMP-13 with an IC₅₀ value of 27.3 nM, GF-02 inhibits MMP-13 with an IC₅₀ value of 8.9 nM, GF-03 inhibits MMP-13 with an IC₅₀ value of 61.8 nM, while GF-04 inhibits MMP-13 with an IC₅₀ value of 91.0 nM.

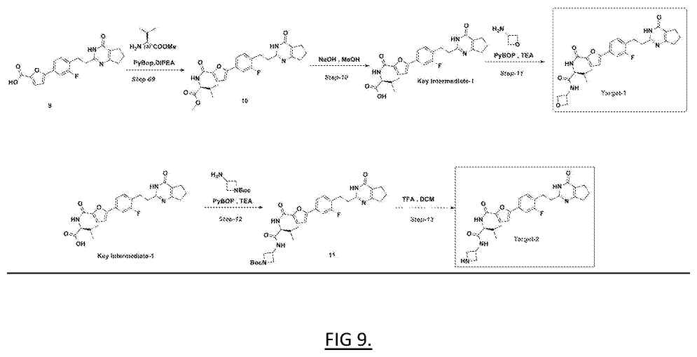

In Scheme 2 (FIG. 9), assembly of GF-01 (Target-1) and GF-03 (Target-2) is described.

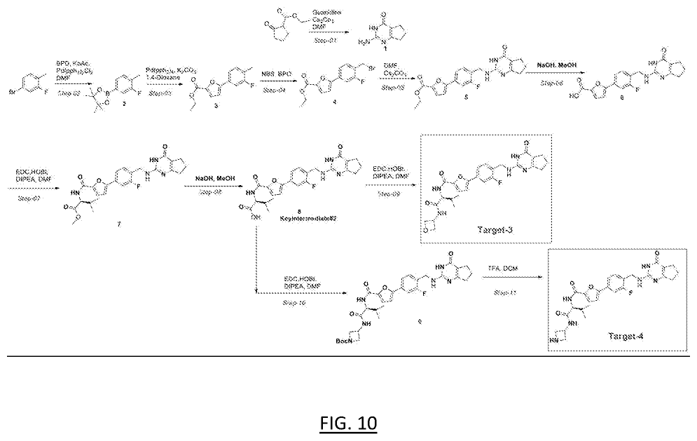

In Scheme 3 (FIG. 10), assembly of GF-02 (Target-3) and GF-04 (Target-4) is described.

In some embodiments of an MMP-13 inhibitor, the MMP-13 inhibitor has one of the following formulas:

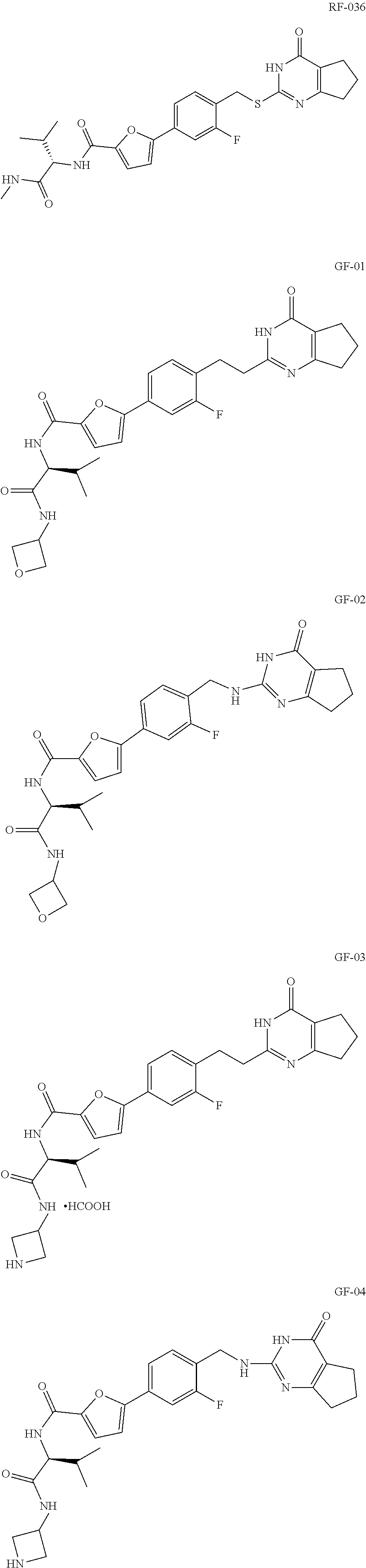 [SMILES: CNC(=O)[C@@H](NC(=O)c1ccc(-c2ccc(CSc3nc4c(c(=O)[nH]3)CCC4)c(F)c2)o1)C(C)C ; CC(C)[C@H](NC(=O)c1ccc(-c2ccc(CCc3nc4c(c(=O)[nH]3)CCC4)c(F)c2)o1)C(=O)NC1COC1 ; CC(C)[C@H](NC(=O)c1ccc(-c2ccc(CNc3nc4c(c(=O)[nH]3)CCC4)c(F)c2)o1)C(=O)NC1COC1 ; CC(C)[C@H](NC(=O)c1ccc(-c2ccc(CCc3nc4c(c(=O)[nH]3)CCC4)c(F)c2)o1)C(=O)NC1CNC1 ; "·HCOOH" ; CC(C)[C@H](NC(=O)c1ccc(-c2ccc(CNc3nc4c(c(=O)[nH]3)CCC4)c(F)c2)o1)C(=O)NC1CNC1]

Methods of making the MMP-13 inhibitor compounds are further described below in the Examples. Compositions containing one MMP-13 inhibitor compound typically contain a sufficient amount of the one MMP-13 inhibitor for inhibiting multiple myeloma cell growth. Compositions including a compound according to any embodiments described herein typically also include a pharmaceutically acceptable carrier. The compounds and compositions described herein may be administered to an individual (e.g., rodents, humans, nonhuman primates, canines, felines, ovines, equines, bovines, insects) in any suitable formulation according to conventional pharmaceutical practice (see, e.g., Remington: The Science and Practice of Pharmacy (21st ed.), ed. A. R. Gennaro, Lippincott Williams & Wilkins, (2005) and Encyclopedia of Pharmaceutical Technology, (3^{rd} ed.) eds. J. Swarbrick and J. C. Boylan, Marcel Dekker, CRC Press, New York (2006), a standard text in this field, and in USP/NF). For example, a composition including an MMP-13 inhibitor may be formulated in pharmaceutically acceptable carriers or diluents such as physiological saline or a buffered salt solution. Suitable carriers and diluents can be selected on the basis of mode and route of administration and standard pharmaceutical practice. A description of exemplary pharmaceutically acceptable carriers and diluents, as well as pharmaceutical formulations, can be found in Remington: supra. Other substances may be added to the compounds and compositions to stabilize and/or preserve them.

The compounds and compositions described herein may be administered to an individual (e.g., a mammal) by any conventional technique. Typically, such administration will be parenteral (e.g., intravenous, subcutaneous, intramuscular, intraperitoneal, oral, nasal, or intrathecal introduction). The compositions may also be administered directly to a target site (e.g., bone marrow). The compositions may be administered in a single bolus, multiple injections, or by continuous infusion (e.g., intravenously, by peritoneal dialysis, pump infusion). For parenteral administration, the compositions are preferably formulated in a sterilized pyrogen-free form.

In some embodiments, an MMP-13 inhibitor or composition as described herein may be in a form suitable for oral administration or sterile injection. To prepare a composition for sterile injection, the suitable active therapeutic agent(s) (e.g., a therapeutically effective amount of one MMP-13 inhibitor) is dissolved or suspended in a parenterally acceptable liquid vehicle. Among acceptable vehicles and solvents that may be employed are water, water adjusted to a suitable pH by addition of an appropriate amount of hydrochloric acid, sodium hydroxide or a suitable buffer, 1,3-butanediol, Ringer's solution, and isotonic sodium chloride solution and dextrose solution (D5W, 0.9% sterile saline). The aqueous formulation may also contain one or more preservatives (e.g., methyl, ethyl, or n-propyl p-hydroxybenzoate). In cases where the therapeutic agent (one MMP-13 inhibitor) is only sparingly or slightly soluble in water, a dissolution enhancing or solubilizing agent can be added, or the solvent may include 10-60% w/w of propylene glycol or the like.

### Methods of Treating Multiple Myeloma

In the experiments described below, systemic ablation of host MMP-13 significantly mitigated cancer associated bone disease. Methods of treating multiple myeloma in an individual include administering to the individual an MMP-13 inhibitor as described herein or a composition including one MMP-13 inhibitor as described herein in a therapeutically effective amount to reduce MMP-13 proteolytic activity in the individual. In some embodiments, the individual is considered high-risk for progression of the multiple myeloma. An individual to be treated includes any individual who has any stage of multiple myeloma. In some embodiments, the MMP-13 inhibitor is RF-036:

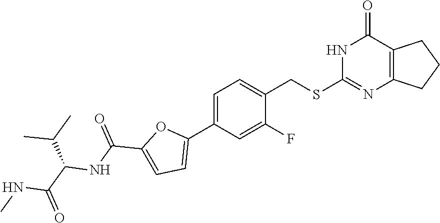 [SMILES: CNC(=O)[C@@H](NC(=O)c1ccc(-c2ccc(CSc3nc4c(c(=O)[nH]3)CCC4)c(F)c2)o1)C(C)C]

In the experiments described below, RF-036 effectively limited multiple myeloma viability and osteoclastogenesis in vitro and significantly inhibited multiple myeloma burden in vivo. In other embodiments of the methods, the MMP-13 inhibitor is one of:

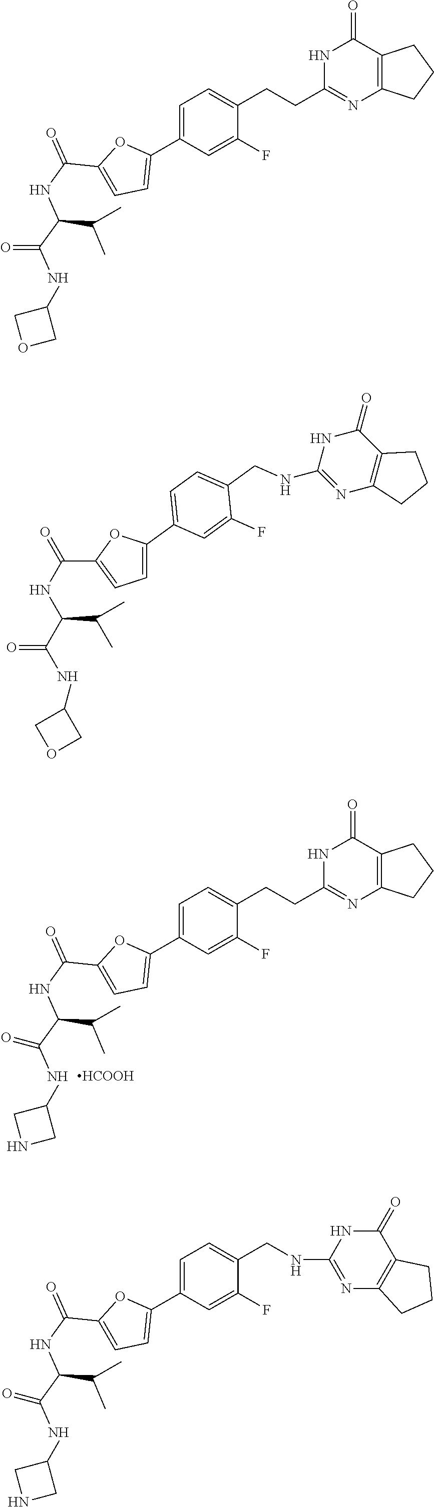 [SMILES: CC(C)[C@H](NC(=O)c1ccc(-c2ccc(CCc3nc4c(c(=O)[nH]3)CCC4)c(F)c2)o1)C(=O)NC1COC1 ; CC(C)[C@H](NC(=O)c1ccc(-c2ccc(CNc3nc4c(c(=O)[nH]3)CCC4)c(F)c2)o1)C(=O)NC1COC1 ; CC(C)[C@H](NC(=O)c1ccc(-c2ccc(CCc3nc4c(c(=O)[nH]3)CCC4)c(F)c2)o1)C(=O)NC1CNC1 ; "·HCOOH" ; CC(C)[C@H](NC(=O)c1ccc(-c2ccc(CNc3nc4c(c(=O)[nH]3)CCC4)c(F)c2)o1)C(=O)NC1CNC1]

In a method of treating multiple myeloma in an individual, administering the compound or composition selectively kills multiple myeloma cells in the individual. Typically, administering the compound or composition reduces growth of multiple myeloma cells in the individual, inhibits multiple myeloma-induced osteoclastogenesis in the individual, and increases survival time in the individual.

Any suitable methods of administering an MMP-13 inhibitor or composition as described herein to an individual may be used. In these methods, the compounds and compositions can be administered to an individual by any suitable route, e.g., oral, buccal (e.g., sub-lingual), and parenteral (e.g., subcutaneous, intramuscular, intradermal, or intravenous) administration. In some embodiments of treating multiple myeloma, as mentioned above, an MMP-13 inhibitor or composition may be administered systemically by intravenous injection. In another embodiment, an MMP-13 inhibitor or composition may be administered directly to a target site, by, for example, surgical delivery to a target site (e.g., bone marrow), or by catheter to a site accessible by a blood vessel.

MMP-13 inhibitors and composition as described herein can be administered as a monotherapy or as part of a combination therapy with any other therapeutic agent in a method of treating multiple myeloma in an individual in need thereof. In some embodiments of a combination therapy, a first composition may include an MMP-13 inhibitor as described herein, and a second composition may include another therapeutic agent. In such embodiments, the first composition may be administered at the same time point or approximately the same time point as the second composition. Alternatively, the first and second compositions may be administered at different time points. Combinations are expected to be advantageously synergistic. Therapeutic combinations that specifically inhibit multiple myeloma-induced osteoclastogenesis and/or reduce MMP-13 concentration and/or MMP-13 proteolytic activity are identified as useful in the methods described herein. For example, an MMP-13 inhibitor as described herein can be administered with one or more of bortezomib/carfilzomib, melphalan, and a bisphosphonate(s).

The therapeutic methods described herein in general include administration of a therapeutically effective amount of one MMP-13 inhibitor and compositions described herein to an individual (e.g., human) in need thereof, particularly a human. Such treatment will be suitably administered to individuals, particularly humans, suffering from, having, susceptible to, or at risk for a disease, disorder, or symptom thereof (e.g., multiple myeloma). Determination of those individuals “at risk” can be made by any objective or subjective determination by a diagnostic test or opinion of a subject or health care provider.

### Effective Doses

The MMP-13 inhibitors and compositions described herein are preferably administered to an individual in need thereof (e.g., human having multiple myeloma) in an effective amount, that is, an amount capable of producing a desirable result in a treated individual. Desirable results include one or more of, for example, selectively killing multiple myeloma cells in the individual, reducing growth of multiple myeloma cells in the individual, inhibiting multiple myeloma-induced osteoclastogenesis in the individual, and prolonging survival of the individual. Such a therapeutically effective amount can be determined according to standard methods. Toxicity and therapeutic efficacy of the MMP-13 inhibitors and compositions utilized in the methods described herein can be determined by standard pharmaceutical procedures. As is well known in the medical and veterinary arts, dosage for any one individual depends on many factors, including the individuals size, body surface area, age, the particular composition to be administered, time and route of administration, general health, and other drugs being administered concurrently. A delivery dose of an MMP-13 inhibitor as described herein is determined based on preclinical efficacy and safety.

### Kits

Described herein are kits for treating multiple myeloma in an individual (e.g., human). A typical kit includes a composition including one MMP-13 inhibitor as described herein and a pharmaceutically acceptable carrier, and instructions for use. Kits also typically include a container and packaging. Instructional materials for preparation and use of the kit components are generally included. While the instructional materials typically include written or printed materials, they are not limited to such. Any medium capable of storing such instructions and communicating them to an end user is encompassed by the kits herein. Such media include, but are not limited to electronic storage media (e.g., magnetic discs, tapes, cartridges, chips), optical media (e.g., CD ROM), and the like. Such media may include addresses to internet sites that provide such instructional materials.

### EXAMPLES

The present invention is further illustrated by the following specific examples. The examples are provided for illustration only and should not be construed as limiting the scope of the invention in any way.

### Example 1—Inhibition of Multiple Myeloma

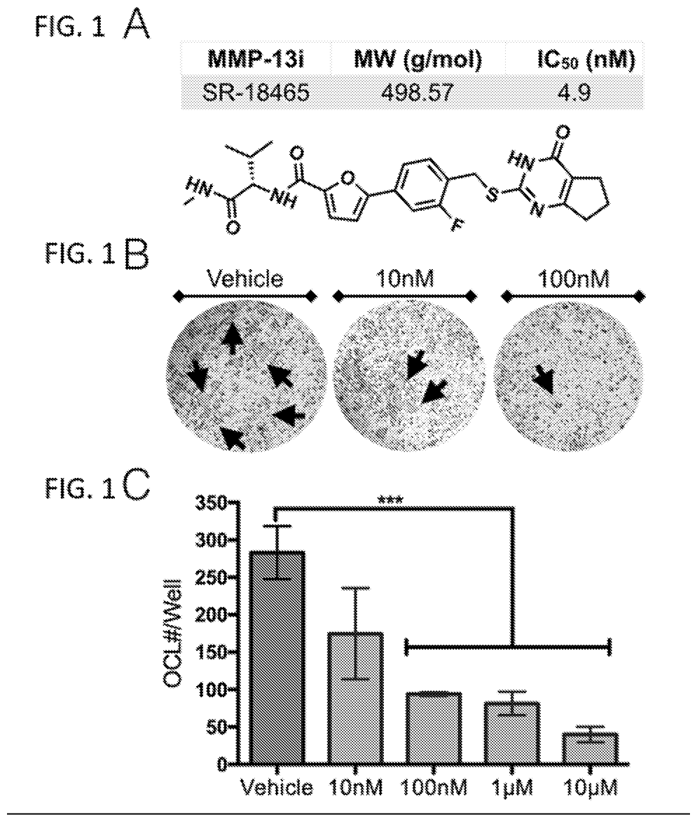

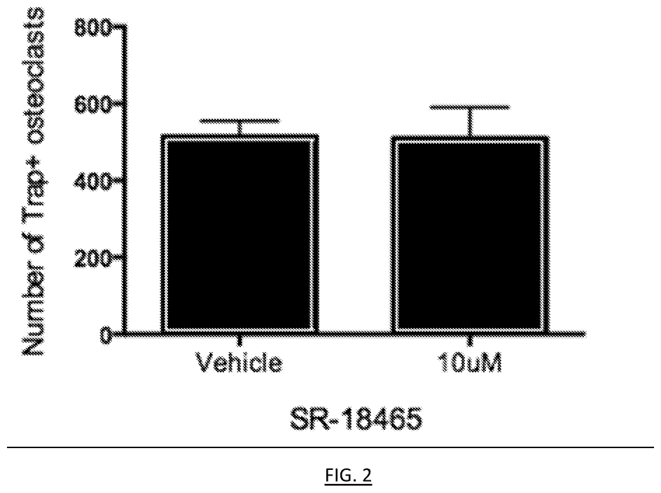

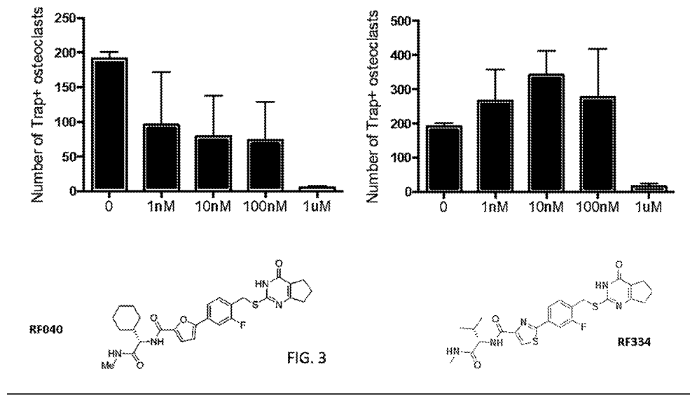

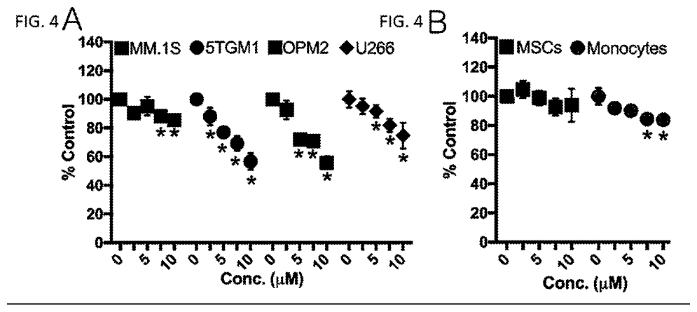

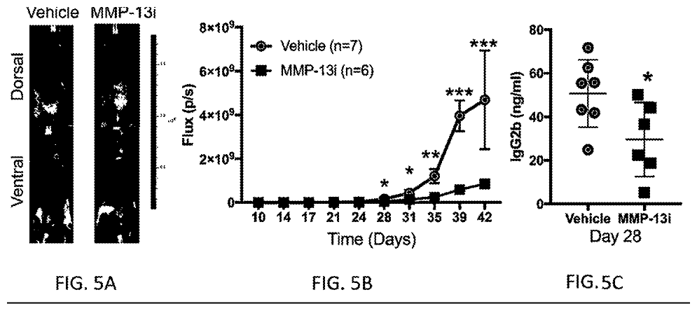

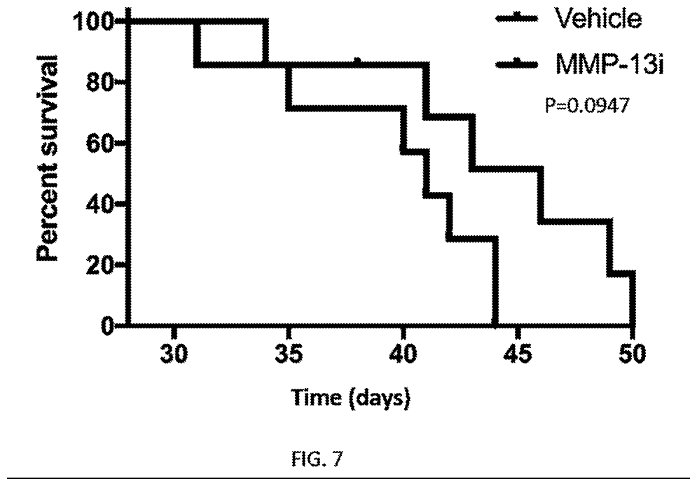

MMP-13 inhibitor RF-036 was shown to be highly selective for MMP-13 with an IC₅₀ of 13 nM compared to MMP-1 (5 μM), MMP-2 (730 nM), MMP-8 (600 nM), MMP-9 (>10 μM), and MT1-MMP/MMP-14 (>10 μM) (compound (S)-17b in Choi et al., J Med Chem (2017)60:3814-3827). RF-036 significantly mitigated osteoclast formation in bone marrow co-cultures that contained bone MSCs (FIG. 1). MMP-13 activity is thus important for osteoclastogenesis. RF-036 was not simply cytotoxic, as treatment of mature osteoclasts with RF-036 has no effect on cell viability (FIG. 2). The inhibition of osteoclastogenesis was not a general property of all MMP-13 inhibitors, as RF-040 (compound (S)-17c in Choi et al., J Med Chem (2017)60:3814-3827) behaved similarly to RF-036 (FIG. 3, left panel) while RF-334 (compound 52 in Fuerst et al., Bioorg Med Chem (2018)26:4984-4995) exhibited much lower activity then RF-036 (FIG. 3, right panel). It was observed that RF-036 directly impacts the viability of several multiple myeloma cell lines by up to 50% over 48 h (FIG. 4A) but has minimal effects on stromal cells of the bone microenvironment, such as MSCs and monocytes (FIG. 4B). In vivo, RF-036 significantly reduces the growth of multiple myeloma (FIG. 5) and increases overall survival in multiple myeloma bearing mice (FIG. 7). Due to solubility issues, only 1/10^{th} of the recommended dose was administered to the animals. Subsequently, solubilization of the drug was optimized with a new vehicle formulation that allows administration of 20 mg/kg. In this formulation, RF-036 is first fully dissolved in dimethyl sulfoxide (DMSO), then the solution is added to an aqueous environment. RF-036 effectively limits myeloma and osteoclast viability in vitro and significantly reduces myeloma burden in vivo.

In addition to RF-036, a class of selective MMP-13 inhibitors that modulates multiple myeloma-induced osteoclastogenesis is of the formula:

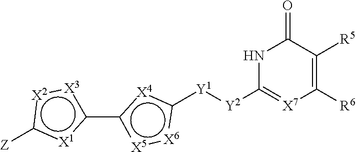

wherein

group Z is of formula C(═O)NHCH(R^{2A})C(═O)NHR^{2B};

R^{2A} is (C₁-C₄)alkyl or (C₃)cycloalkyl, and R^{2B} is 4-membered heterocyclyl;

X¹ is O;

X² and X³ are each independently CR³;

such that the ring comprising X¹, X², and X³ is heteroaryl;

R³ is independently at each occurrence H;

X⁴ is C(R⁴)═C(R⁴);

X⁵ and X⁶ are each independently CR⁴;

such that the ring comprising X⁴, X⁵, and X⁶ is aryl;

R⁴ is independently at each occurrence H or F;

Y¹ is CHR;

Y² is S, CHR, or NR;

X⁷ is N;

R is H;

R⁵ and R⁶ together with the ring carbon atoms to which they are bonded together form a 5-membered cycloalkyl ring;

or a pharmaceutically acceptable salt thereof.

For instance, X⁵ and X⁶ can both be CR⁴.

For instance, X¹ can be O and X⁴ can be CH═CH.

For instance, X¹ can be O, X² and X³ can both be CH; and R⁴ can be H or F.

### Example 2—MMP-13 Contributes to Multiple Myeloma Progression and Ablation of MMP-13 Improves Survival

To test whether host derived MMP-13 contributed to multiple myeloma progression, immunocompromised recombinase activating gene-2 (RAG-2) MMP-13 double null animals were generated that are receptive to engraftment with the murine multiple myeloma cell line 5TGM1 (Fowler et al., Dis Model Mech. (2009)2:604-611). In wild type mice MMP-13 staining was largely confined to bone lining cells and cement lines while no staining was observed in MMP-13 null tissues. RT-PCR confirmed tissue analyses showing MMP-13 expression by stromal cells but not by osteoclasts.

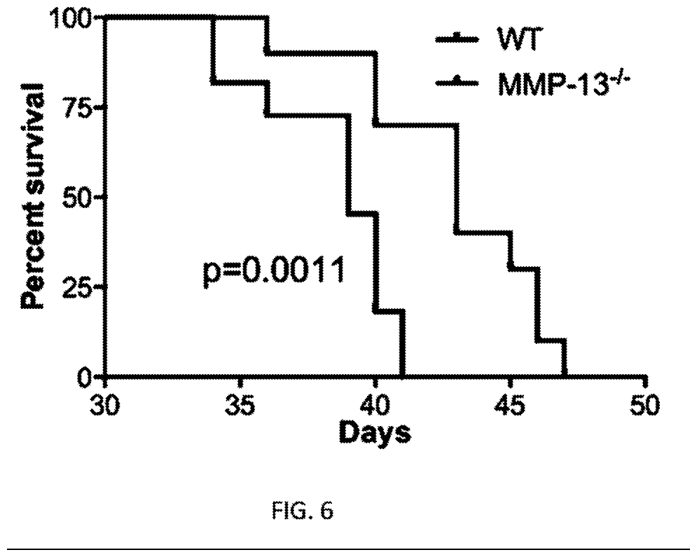

Wild type or MMP-13 null animals (n=10/group) were inoculated with luciferase expressing 5TGM1 cells. Growth was measured weekly (bioluminescence and serum IgG2B levels). Surprisingly, despite no apparent difference in growth rates, it was found that overall survival in the MMP-13 null multiple myeloma bearing mice was significantly higher than controls with median survival times of 43 and 39 days, respectively (p=0.0011; FIG. 6). This difference is impressive given the rapidity and aggressiveness of the 5TGM1 model. Furthermore, the observed difference in overall survival time is in keeping with standard of care therapies tested in the 5TGM1 model and the related 5T2MM model such as bortezomib, bisphosphonates and melphalan.

### Example 3—Evaluating MMP-13 Inhibitor Efficacy

The data described above in Example 1 show the efficacy of a highly selective inhibitor of MMP-13 in limiting multiple myeloma cell growth and osteoclastogenesis in vitro and in vivo. To evaluate MMP-13 inhibitor efficacy in human multiple myeloma ex vivo, the effect of MMP-13 inhibition on the viability of ex vivo isolated myeloma cells from de-identified cancer center patients that are newly diagnosed (n=50) is examined. Briefly, CD138 myeloma cells are isolated from de-identified patient bone marrow aspirates. The isolated cells remain viable for approximately 5 days with an average of >107 myeloma cells isolated per biopsy. A portion of these cells are used to assess MMP-13 expression (PCR/immunoblot) while MMP-13 activity is determined with a selective near infrared MMP-13 beacon. The remainder of the CD138 cells are used to determine the impact of MMP-13 inhibition on cell viability. Myeloma cells (4×10³) are seeded into each well of a 384-well plate. Cells are then treated with the MMP-13 inhibitor (e.g., RF-036; 0.1 nM to 10 μM) and viability is determined. A high throughput platform is also used to determine whether the MMP-13 inhibitor acts synergistically with standard of care inhibitors such as bortezomib, carfilzomib, melphalan and bisphosphonates. Statistical analyses with ex vivo patient samples are performed.

The efficacy of any candidate MMP-13 inhibitor compound(s) is evaluated in vivo. Given that MMP-13 inhibition can impact myeloma viability and osteoclast formation (FIG. 1) and that bisphosphonates impact the viability of mature bone resorbing osteoclasts, whether MMP-13 inhibition has an additive/synergistic effect when combined with bisphosphonates is also determined. To this end, 5TGM1 (1×10⁶) and U266 (5×10⁶) luciferase expressing cells are inoculated into 6-8 week old RAG-2 null mice by tail vein injection (n=10 per group). After one week, mice are imaged and randomized and the treatments initiated. Four treatment groups are used, 1) Vehicle (0.1% DMSO), 2) MMP-13 inhibitor (e.g., RF-036), 3) bisphosphonate (zoledronic acid, 0.1 mg/kg, 3×week sub-cutaneously) and 4) MMP-13 inhibitor and bisphosphonate. Longitudinal imaging and post study analyses are performed. Peripheral blood is collected on a weekly basis and bone marrow supernatants are collected at the study endpoint for MMP-13 activity assays.

### Example 4—Treatment with RF-036 Increases Survival Time

The overall survival time of 5TGM1 multiple myeloma bearing mice treated with RF-036 (MMP-13i) (n=6; 2 mg/kg daily) was compared to vehicle control (n=7) (FIG. 7). The median survival time for the RF-036 group was 46 days compared to 41 for the vehicle control group.

### Other Embodiments

Any improvement may be made in part or all of the compounds, compositions, kits and method steps. All references, including publications, patent applications, and patents, cited herein are hereby incorporated by reference. The use of any and all examples, or exemplary language (e.g., “such as”) provided herein, is intended to illuminate the invention and does not pose a limitation on the scope of the invention unless otherwise claimed. Any statement herein as to the nature or benefits of the invention or of the preferred embodiments is not intended to be limiting, and the appended claims should not be deemed to be limited by such statements. More generally, no language in the specification should be construed as indicating any non-claimed element as being essential to the practice of the invention. This invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contraindicated by context.

1. A compound of Formula A: 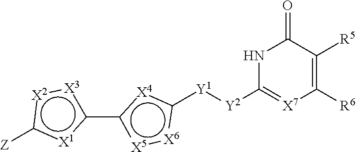
wherein
group Z is of formula C(═O)NHCH(R^{2A})C(═O)NHR^{2B};
R^{2A} is (C₁-C₄)alkyl or (C₃)cycloalkyl, and R^{2B} is 4-membered heterocyclyl or CH₃;
X¹ is O;
X² and X³ are each independently CR³;
such that the ring comprising X¹, X², and X³ is heteroaryl;
R³ is independently at each occurrence H;
X⁴ is C(R⁴)═C(R⁴);
X⁵ and X⁶ are each independently CR⁴;
such that the ring comprising X⁴, X⁵, and X⁶ is aryl;
R⁴ is independently at each occurrence H or F;
Y¹ is CHR;
Y² is S, CHR, or NR;
X⁷ is N;
R is H;
R⁵ and R⁶ together with the ring carbon atoms to which they are bonded together form a 5-membered cycloalkyl ring;
or a pharmaceutically acceptable salt thereof.

2. The compound of claim 1, wherein X⁵ and X⁶ are both CR⁴.

3. The compound of claim 1, wherein X¹ is O and X⁴ is CH═CH.

4. The compound of claim 1, wherein X¹ is O, X² and X³ are both CH, and R⁴ is H or F.

5. The compound of claim 1, having the formula:

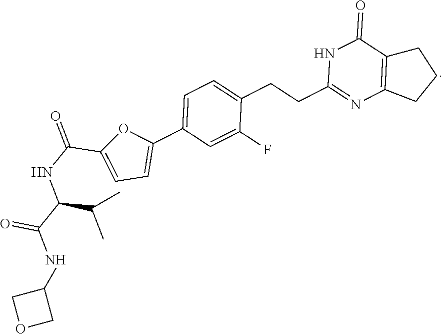 [SMILES: CC(C)[C@H](NC(=O)c1ccc(-c2ccc(CCc3nc4c(c(=O)[nH]3)CCC4)c(F)c2)o1)C(=O)NC1COC1]

6. The compound of claim 1, having the formula:

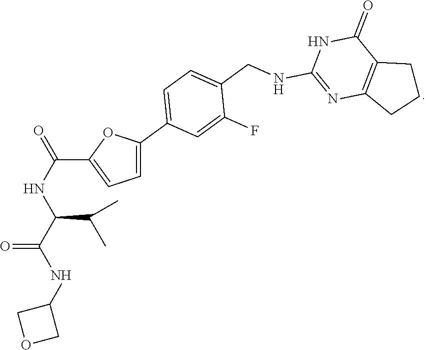 [SMILES: CC(C)[C@H](NC(=O)c1ccc(-c2ccc(CNc3nc4c(c(=O)[nH]3)CCC4)c(F)c2)o1)C(=O)NC1COC1]

7. The compound of claim 1, having the formula:

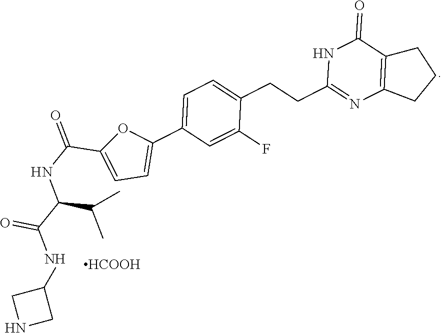 [SMILES: CC(C)[C@H](NC(=O)c1ccc(-c2ccc(CCc3nc4c(c(=O)[nH]3)CCC4)c(F)c2)o1)C(=O)NC1CNC1 ; "·HCOOH"]

8. The compound of claim 1, having the formula:

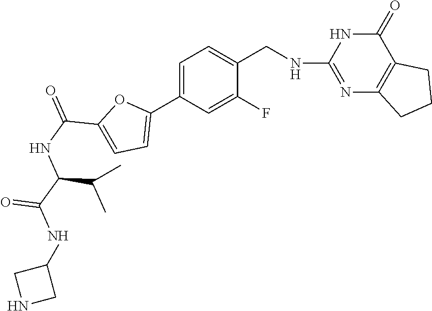 [SMILES: CC(C)[C@H](NC(=O)c1ccc(-c2ccc(CNc3nc4c(c(=O)[nH]3)CCC4)c(F)c2)o1)C(=O)NC1CNC1]

9. The compound of claim 1, wherein the compound inhibits multiple myeloma cell growth.

10. A composition comprising the compound of claim 1 and a pharmaceutically acceptable carrier.

11. A composition comprising the compound of claim 5 and a pharmaceutically acceptable carrier.

12. A composition comprising the compound of claim 6 and a pharmaceutically acceptable carrier.

13. A composition comprising the compound of claim 7 and a pharmaceutically acceptable carrier.

14. A composition comprising the compound of claim 8 and a pharmaceutically acceptable carrier.

15. A method of treating multiple myeloma in an individual comprising administering to the individual a compound according to claim 1 in a therapeutically effective amount to reduce at least one of: MMP-13 concentration and MMP-13 proteolytic activity in the individual.

16. The method of claim 15, wherein the compound has the formula:

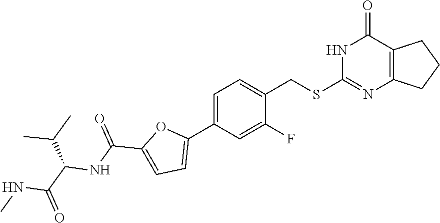 [SMILES: CNC(=O)[C@@H](NC(=O)c1ccc(-c2ccc(CSc3nc4c(c(=O)[nH]3)CCC4)c(F)c2)o1)C(C)C]

17. The method of claim 15, wherein administering the compound or composition selectively kills multiple myeloma cells in the individual.

18. The method of claim 15, wherein administering the compound or composition reduces growth of multiple myeloma cells in the individual.

19. The method of claim 15, wherein administering the compound or composition inhibits multiple myeloma-induced osteoclastogenesis in the individual.

20. The method of claim 15, wherein administering the compound or composition increases survival time in the individual.

21. The method of claim 15, wherein the individual is considered high-risk for progression of the multiple myeloma.

22. The method of claim 15, wherein the individual is a human.

23. The method of claim 15, further comprising detecting a state or condition of multiple myeloma in the individual prior to administering the compound or the composition to the individual.
